(19) United States
(12) Patent Application Publication
(10) Pub. No.: US 2015/0173618 A1
(43) Pub. Date: Jun. 25, 2015

(54) OPTICAL BRAIN-FUNCTION MEASUREMENT APPARATUS

(71) Applicant: Panasonic Intellectual Property Corporation of America, Torrance, CA (US)

(72) Inventor: KOICHI KUSUKAME, Nara (JP)

(21) Appl. No.: 14/570,022

(22) Filed: Dec. 15, 2014

(30) Foreign Application Priority Data
Dec. 20, 2013 (JP) 2013-264297

(51) Int. Cl. A61B 5/00; A61B 5/1455

(52) U.S. Cl. CPC A61B 5/0064; A61B 5/4064; A61B 5/14553

(57) ABSTRACT

An optical brain-function measurement apparatus includes a light source unit that generates infrared light to be radiated onto a human head; a detection unit that detects the infrared light which is diffusely-reflected within the human head and which is exited from one or more head position; and an optical system that guides the infrared light emitted from the light source unit toward the human head and that controls an infrared-light irradiation position on a surface of the human head. At least one of the light source unit and the detection unit is of a non-contact type. The one or more head positions detected by the detection unit include at least one position that is different from the infrared-light irradiation position controlled by the optical system.

### CROSS REFERENCES TO RELATED APPLICATIONS

This application claims priority to Japanese Patent Application No. 2013-264297, filed on Dec. 20, 2013, the contents of which are hereby incorporated by reference.

### BACKGROUND

1. Technical Field

The present disclosure relates to optical brain-function measurement apparatuses that noninvasively measure brain activity by using infrared light so as to measure brain function.

2. Description of the Related Art

Near-infrared light (wavelength: 700 nm to 1000 nm) has relatively high transmissivity with respect to biological tissues, such as muscles, bones and lipid, and has characteristics in which the near-infrared light is absorbed by oxyhemoglobin and deoxyhemoglobin within the blood. Therefore, as discussed in Japanese Unexamined Patent Application Publication No. 2012-223523, near-infrared spectroscopy (referred to as “NIRS” hereinafter) that utilizes these characteristics is used for measuring a change in blood flow.

For example, as discussed in Japanese Unexamined Patent Application Publication No. 2012-223523, an optical brain-function measurement apparatus that measures brain function by using this NIRS has an optical transmission-reception probe that radiates near-infrared light onto the head, receives light diffusely-reflected by the brain, detects this light, and measures the concentration of oxyhemoglobin and deoxyhemoglobin within the blood flowing through the brain based on this measurement result so as to measure the state of brain activity (brain function) based on the oxygen state of hemoglobin. Furthermore, this optical brain-function measurement apparatus has a main device such as a personal computer that manages the measurement result.

FIG. 10 illustrates an optical brain-function measurement apparatus in the related art. As shown in FIG. 10, the optical brain-function measurement apparatus in the related art includes a wearable member 201 that is to be worn on the head. Moreover, a light source unit 202 that emits near-infrared light toward the head and a detection unit 203 that receives the near-infrared light diffusely-reflected by the brain so as to detect this light are attached to the wearable member 201. The light source unit 202 and the detection unit 203 are connected to a main device 205 via wires 204, such as optical fibers or electric wires.

Accordingly, in the optical brain-function measurement apparatus in the related art, since the light source unit 202 and the detection unit 203 are secured to the head, a position where the infrared light enters the head (referred to as “light entrance position” hereinafter) and a position where the light exiting the head is guided to the detection unit 203 (referred to as “light exit position” hereinafter) are fixed.

### SUMMARY

An optical brain-function measurement apparatus according to an aspect of the present disclosure includes a light source unit that generates infrared light to be radiated onto a human head; a detection unit that detects the infrared light which is diffusely-reflected within the human head and which is exited from one or more head position; and an optical system that guides the infrared light emitted from the light source unit toward the human head and that controls an infrared-light irradiation position on a surface of the human head. The light source unit is a light source of a non-contact type that does not contact to the human or the detection unit is a detector of a non-contact type that does not contact to the human. The one or more head positions detected by the detection unit include at least one position that is different from the infrared-light irradiation position controlled by the optical system.

According to the present disclosure, an optical brain-function measurement apparatus that can measure brain function at an arbitrary position of the human head can be provided.

### BRIEF DESCRIPTION OF THE DRAWINGS

FIG. 1 schematically illustrates an example of an optical brain-function measurement apparatus according to a first embodiment of the present disclosure.

FIG. 2 is a block diagram illustrating an example of an optical brain-function measurement apparatus according to the present disclosure.

FIG. 3 schematically illustrates another example of an optical brain-function measurement apparatus according to the first embodiment of the present disclosure.

FIG. 4 schematically illustrates another example of an optical brain-function measurement apparatus according to the first embodiment of the present disclosure.

FIG. 5 schematically illustrates an example of an optical brain-function measurement apparatus according to a second embodiment of the present disclosure.

FIG. 6 schematically illustrates another example of an optical brain-function measurement apparatus according to the second embodiment of the present disclosure.

FIG. 7 schematically illustrates an example of an optical brain-function measurement apparatus according to a third embodiment of the present disclosure.

FIG. 8 schematically illustrates another example of an optical brain-function measurement apparatus according to the third embodiment of the present disclosure.

FIG. 9 schematically illustrates an example of an optical brain-function measurement apparatus according to a fourth embodiment of the present disclosure.

FIG. 10 schematically illustrates an optical brain-function measurement apparatus in the related art.

### DETAILED DESCRIPTION

First, the matters studied by the present inventors for achieving the aspects of the present disclosure will be described.

### Grounds for Present Disclosure

In the optical brain-function measurement apparatus in the related art having the light source unit 202 and the detection unit 203 that are attached to the wearable member 201 to be worn on the head, once the wearable member 201 is worn on the head, it is difficult to change the positions of the light source unit 202 and the detection unit 203. This is a problem in that the optical brain-function measurement apparatus is not suitable for measuring the brain function at an arbitrary position.

The present inventors have realized the problem in that the measurement result varies depending on the light entrance position and the light exit position in the optical brain-function measurement apparatus in the related art, and have studied the reasons therefor.

As a result of the study, because an area with a particularly high blood flow, such as an artery or a vein, exists in the scalp (or also called the surface of the head), the present inventors have realized that the light from the light source unit 202 attenuates when the light passes through this area. Thus, when a high-blood-flow area exists near at least one of the light entrance position and the light exit position, the light is absorbed by the high-blood-flow area near that position so that the light attenuation rate from the light source unit 202 to the detection unit 203 changes. The present inventors have realized that this is a problem in that the sensitivity of the optical brain-function measurement apparatus significantly changes (particularly, the sensitivity becomes insufficient at the high-blood-flow area).

In order to solve this problem, the present inventors have conceived that a significant change in the sensitivity of the optical brain-function measurement apparatus needs to be suppressed (or eliminated) by adjusting the light entrance position or the light exit position. This may be achieved by making at least one of the light entrance position or the light exit position positionally adjustable so that the effect of the light from the light source unit 202 being significantly absorbed by the high-blood-flow area at the surface of the human head is reduced (or eliminated).

The present disclosure provides an optical brain-function measurement apparatus that achieves brain-function measurement at an arbitrary position of the human head.

In the following description, a first embodiment relates to a case where both the light entrance position and the light exit position are positionally adjustable, a second embodiment relates to a case where only the light exit position is adjustable, and a third embodiment relates to a case where only the light entrance position is adjustable.

In the first to third embodiments of the present disclosure, at least one of the light entrance position and the light exit position can be located away from an artery or a vein of the scalp. For example, by performing measurement while changing the position of one of the light entrance position and the light exit position, if the sensitivity (i.e., light quantity measured by a detection unit) significantly changes particularly at a short distance of about 2 mm to 0.5 mm, it can be determined that an artery or a vein exists under a part of the scalp corresponding to one of the light entrance position and the light exit position that has been positionally changed.

Furthermore, without having to determine the positions of arteries and veins, a measurement result that is not affected by artery distribution and vein distribution can be obtained by averaging measurement results from a plurality of light entrance positions and light exit positions.

Embodiments to be described below indicate specific examples of the present disclosure. Numerical values, shapes, components, steps, the order of steps, and so on in the following embodiments are examples and are not intended to limit the present disclosure. Furthermore, of the components in the following embodiments, components that are not defined in the independent claim indicating the broadest concept are described as arbitrary components. Moreover, the contents in all of the embodiments may be combined.

### First Embodiment

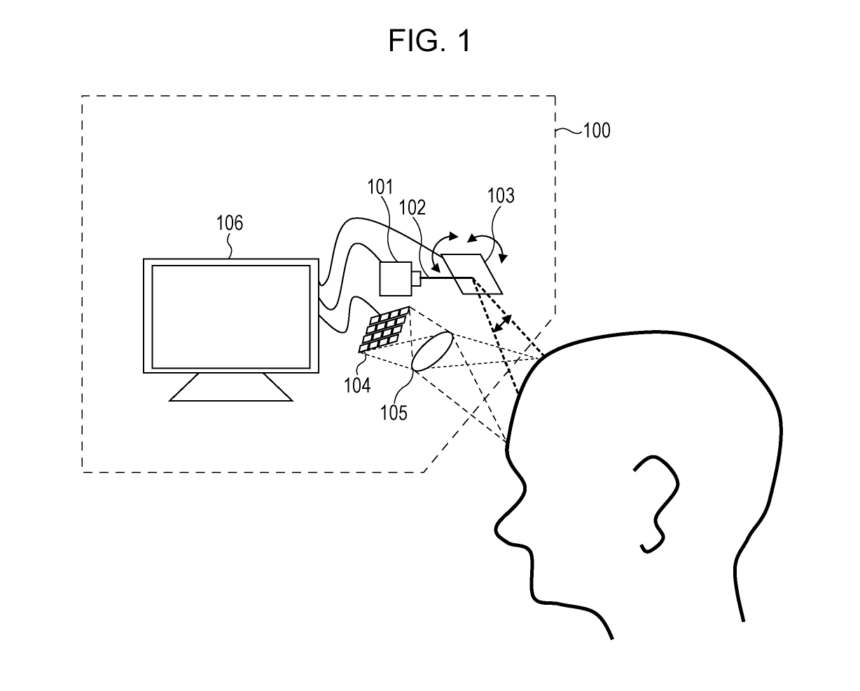

FIG. 1 illustrates an example of an optical brain-function measurement apparatus 100 according to a first embodiment.

As shown in FIG. 1, the optical brain-function measurement apparatus 100 includes a light source unit 101, an optical-path changing unit 103, a detection unit 104, an optical system 105, and a main device 106.

The light source unit 101 emits infrared light 102. The infrared light 102 is radiated onto the human head. The infrared light 102 is guided to the human head by an optical system disposed in an optical path of the infrared light 102 (between the light source unit 101 and the head). In the example shown in FIG. 1, the optical system that guides the infrared light 102 to the human head includes, for example, the optical-path changing unit 103 that changes the optical path of the infrared light 102. The optical path of the infrared light 102 is changed by the optical-path changing unit 103 so that the infrared light 102 is guided onto the human head. If the optical-path changing unit 103 is a mirror, the irradiation position (i.e., the light entrance position) of the infrared light 102 can be shifted by changing the angle of the mirror.

The infrared light 102 radiated onto the human head is diffusely-reflected within the human head and exits the head from a light exit position and the periphery thereof. Regarding the infrared light 102 diffusely-reflected within the human head, if the distance between the light exit position, from which the light exits the head, and the light entrance position is short to a certain extent, the infrared light exiting from that light exit position (referred to as “first exit position” hereinafter) contains a high proportion of infrared light mainly passing through the scalp before exiting from the first exit position. On the other hand, regarding the infrared light 102 diffusely-reflected within the human head, as the distance between the light exit position, from which the light exits the head, and the light entrance position increases, the infrared light exiting from that light exit position (referred to as “second exit position” hereinafter) contains a high proportion of infrared light mainly passing through the scalp, the cranium or the brain, and then the scalp before exiting from the second exit position.

As described above, the infrared light 102 diffusely-reflected within the human head exits the head from a plurality of exit positions and is guided to the detection unit 104 by the optical system 105.

The detection unit 104 includes a plurality of detection elements and measures the intensities (or the light quantities) of infrared light beams exiting from a plurality of locations (i.e., a plurality of light exit positions) on the surface of the head. For example, the detection unit 104 includes detection elements that are two-dimensionally arranged in the vertical and horizontal directions.

It is desirable that the light quantity (i.e., second light quantity) of the infrared light exiting from the second exit position be measured and that the brain function be measured by using the measured light quantity. However, since the infrared light exiting from the second exit position passes through the scalp, as described above, the infrared light is affected by the scalp. On the other hand, by measuring the light quantity (i.e., first light quantity) of the infrared light exiting from the first exit position, the effect on the infrared light when passing through the scalp can be ascertained.

Thus, the light quantity (i.e., second light quantity) of the infrared light exiting from the second exit position and the light quantity (i.e., first light quantity) of the infrared light exiting from the first exit position are both measured. Based on the measured first light quantity, the effect on the infrared light passing through the scalp, which is included in the measured second light quantity, is removed, whereby the brain function can be accurately measured. The calculation for removing the effect on the infrared light passing through the scalp, which is included in the measured second light quantity, based on the measured first light quantity may be performed by, for example, the main device 106 or the detection unit 104.

In this case, since the light quantity of infrared light exiting from a light exit position varies significantly depending on whether or not there is a high-blood-flow area near the light entrance position or the light exit position, the brain function cannot be accurately measured.

The existence of a high-blood-flow area near the light entrance position or the light exit position implies that, for example, there is an artery or a vein existing under an area of the scalp that corresponds to the light entrance position or the light exit position.

In the first embodiment, at least one of the light entrance position and the light exit position is adjusted to be located away from an artery or a vein of the scalp so that the effect on the infrared light passing through the scalp, which is included in the measured second light quantity, can be reduced.

Although the light exit position of infrared light detected by each detection element is fixed in FIG. 1, a desired light exit position is selectable by selecting the corresponding detection element.

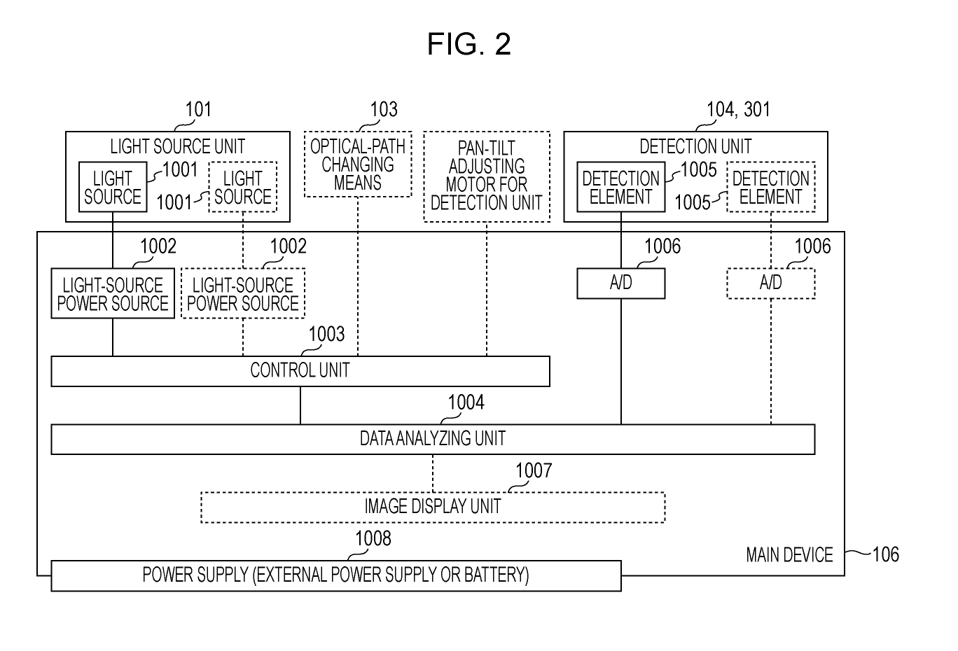

FIG. 2 is a block diagram illustrating an example of an optical brain-function measurement apparatus according to the present disclosure.

As shown in FIG. 2, light sources 1001 within the light source unit 101 in FIG. 1 are supplied with electric power from light-source power sources 1002 within the main device 106. The light-source power sources 1002 and the optical-path changing unit 103 are controlled by a control unit 1003. In other words, the control unit 1003 controls the input (power) of the infrared light 102 to the head as well as the light entrance position thereof.

The main device 106 includes, for example, a memory (not illustrated) and a processor (not illustrated), such as a central processing unit (CPU).

For example, the CPU reads a control program from the memory and executes the control program. The control unit 1003 is realized by the CPU executing the control program stored in the memory.

Alternatively, a function of the control unit 1003 may be implemented by dedicated hardware circuits (or dedicated hardware circuitry), such as application-specific integrated circuit (ASICs) or field programmable gate arrays (FPGAs).

Information related to light-source input power corresponding to the input of the infrared light 102 and information related to the optical-path change state of the optical-path changing unit 103 (i.e., angle information in a case where the optical-path changing unit 103 is a mirror) corresponding to the light entrance position are transmitted from the control unit 1003 to a data analyzing unit 1004. In this case, the data analyzing unit 1004 calculates the light exit position based on the information related to the optical-path change state.

For example, the CPU reads a data analyzing program from the memory and executes the data analyzing program. The data analyzing unit 1004 is realized by the CPU executing the data analyzing program stored in the memory.

Alternatively, a function of the data analyzing unit 1004 may be implemented by dedicated hardware circuits (or dedicated hardware circuitry), such as ASICs or FPGAs.

Furthermore, an analog signal of electric current (or voltage) detected by each detection element 1005 within the detection unit 104 is transmitted to the data analyzing unit 1004 via a corresponding analog-to-digital converter 1006 and is data-processed as output from the light exit position in the corresponding detection element 1005.

Specifically, the data analyzing unit 1004 stores four pieces of information, namely, the input of the infrared light 102, the light entrance position, the output, and the light exit position (e.g., the first exit position or the second exit position).

Based on the four pieces of information, the data analyzing unit 1004 calculates, for example, light absorption characteristics and scatter characteristics at each part of the head, as well as blood flow distribution and oxyhemoglobin/deoxyhemoglobin ratio distribution within the human head (i.e., calculates the brain function state).

With regard to the above-described calculation method, a method similar to that in the optical brain-function measurement apparatus in the related art may be used.

Although not shown, an amplifying configuration is possible by providing amplifiers between the analog-to-digital converters 1006 and the detection elements 1005.

The main device 106 includes an image display unit 1007. The brain function state calculated by the data analyzing unit 1004 may be displayed on the image display unit 1007 so that a subject can ascertain one's own brain function state.

Furthermore, the main device 106 may be equipped with a battery therein or may use an external power supply.

The configuration of the optical brain-function measurement apparatus according to the present disclosure described above with reference to FIG. 2 is an example. A different configuration that achieves a similar function is also permissible.

With regard to the light source unit 101, for example, a semiconductor laser, a solid-state laser, a fiber laser, a super luminescent diode, or a light-emitting diode (LED) is used. Desirably, a semiconductor laser, a solid-state laser, a fiber laser, or a super luminescent diode is used since this allows for the use of more compact optical-path changing unit 103, so that the entire apparatus can be reduced in size.

Furthermore, the light source unit 101 may be a light source unit that generates light beams of a plurality of wavelengths by, for example, including a plurality of lasers, or may be a light source unit that switches the wavelength of light to be generated. By acquiring a larger number of pieces of information within the brain, a larger number of kinds of brain function states can be determined.

Furthermore, laser beams of a plurality of wavelengths emitted from the light source unit 101 may be in the same optical path. Thus, the transmission characteristics of the light beams of the plurality of wavelengths can be ascertained at the same location within the brain, whereby the brain function state can be measured in accordance with measurement of component distribution within the brain.

Furthermore, at least one of the light beams of the plurality of wavelengths may be a light beam with a wavelength shorter than 805 nm, and at least one of the remaining light beams may be a light beam with a wavelength longer than 805 nm. Thus, spatial distribution of oxygen consumption within the brain can be ascertained based on the concentration distribution of oxyhemoglobin and deoxyhemoglobin within the brain. Consequently, an area with active brain function can be ascertained.

In the first embodiment, for example, two laser sources of different wavelengths, namely, 780 nm and 830 nm, and a dichroic mirror are provided within the light source unit 101. Two laser beams emitted from these two laser sources are combined by the dichroic mirror so as to be emitted along a single optical path.

With regard to the optical-path changing unit 103, for example, a polygonal mirror, a galvanometer mirror, a rotatable prism, or a micro-electro-mechanical-system (MEMS) mirror is used. In particular, a two-axis-scanning-type MEMS mirror is used so that a compact, high-speed brain-function measurement apparatus can be achieved.

The optical system 105 is an optical system, such as a lens or a mirror, which guides light beams exiting from different locations of the human head to the different detection elements 1005. For example, this may be achieved by using an optical system that focuses infrared light exiting from the surface of the head onto each detection element and the periphery thereof.

In a case where the optical system 105 includes a lens, antireflection films against the infrared light 102 may be provided over the entrance and exit surfaces of the lens. In a case where the optical system 105 includes a mirror, an anti-transmission film against the infrared light 102 may be provided over the reflection surface of the mirror. Thus, higher-sensitivity brain-function measurement becomes possible.

Furthermore, the optical system 105 is disposed between the surface of the head and the detection unit 104 and may be an optical system constituted of a single lens or a single mirror or an optical system constituted of a plurality of optical components.

Although not shown, the optical system 105 may include a confocal optical system. Thus, noise caused by infrared light scattered in the space between the surface of the human head and the detection unit 104 can be removed, thereby allowing for a higher-sensitivity optical brain-function measurement apparatus.

Furthermore, although not shown, the optical system 105 may include a polarization plate. Thus, a large portion of the infrared light 102 diffusely-reflected at the surface of the human head can be removed, whereas the infrared light 102 entering the human head more selectively and then re-exiting from the surface of the head can be measured. By reducing the proportion of infrared light diffusely-reflected at the surface of the head, higher-sensitivity brain-function measurement becomes possible.

In a case where a polarization plate is used in the optical system 105, it is desirable that the infrared light radiated onto the human head be linearly-polarized light, so that higher-sensitivity brain-function measurement becomes possible.

Furthermore, the infrared light 102 may be split into two orthogonally-polarized light beams, and the two light beams may be simultaneously measured using at least two detection units. Thus, the entrance position and the exit position of the infrared light 102 as well as the light intensity relationship therebetween can be more accurately measured. Consequently, more accurate brain-function measurement becomes possible.

Furthermore, the optical system 105 may include a filter that only transmits the wavelength of the infrared light 102 emitted from the light source unit 101 so as to prevent light of a wavelength other than that of the infrared light 102 from being guided to the detection unit 104. Thus, higher-sensitivity brain-function measurement becomes possible.

Moreover, on-off control (i.e., output control) of the light source unit 101 may be performed by the control unit 1003 within the main device 106. By comparing the light quantities of the infrared light 102 emitted from the light source unit 101 and measured by each detection element 1005 at different timings (or when the light source unit 101 is turned on and off), light (noise) generated by a light-emitting member other than the light source unit 101 can be removed, so that intensity distribution (on the surface of the human head) of the infrared light 102 emitted from the light source unit 101 can be measured. Thus, higher-sensitivity brain-function measurement becomes possible.

Furthermore, although not shown, the optical brain-function measurement apparatus according to the present disclosure may include an illuminance sensor. By estimating the intensity of ambient light entering the detection unit 104 based on the illuminance of the installation environment and subtracting the estimated intensity from the intensity of the infrared light 102, the intensity of the infrared light 102 entering the detection unit 104 can be determined more accurately. In other words, higher-sensitivity brain-function measurement becomes possible. The illuminance sensor is desirably installed at a position close to the detection unit 104 and is desirably oriented toward a measurement subject (i.e., in the direction in which the infrared light 102 is radiated). Thus, the intensity of ambient light entering the detection unit 104 can be determined more accurately.

The illuminance sensor may be, for example, a photodiode. In the case of the present disclosure, in order to determine the intensity of near-infrared light, an illuminance sensor with high sensitivity in the infrared region is desired.

Furthermore, by pulse-driving the light source unit 101 and performing optical transmission and reception multiple times while changing the detection timing of the detection unit 104 and the light-emission timing of the light source unit 101, the distance between the human head and the light source unit 101 as well as the distance between the human head and the detection unit 104 can be measured. Since the sensitivity of the detection unit 104 varies depending on distance, sensitivity correction based on distance ascertainment allows for more accurate brain-function measurement.

Furthermore, by adjusting the pulse waveform (i.e., peak intensity and pulse width) of the light source unit 101 as well as the detection time of the detection unit 104, the ratio between the infrared light 102 emitted from the light source unit 101 and the ambient light changes. Specifically, by performing optical transmission and reception multiple times in conditions in which the pulse waveform and the detection time vary, the effect of ambient light can be ascertained and corrected more accurately. In other words, brain-function measurement can be performed more accurately.

Furthermore, since the optical-path changing unit 103 is also controlled by the control unit 1003, the entrance position of the infrared light 102 can be changed on the surface of the human head. Thus, a light exit position calculated by the data analyzing unit 1004 is also changed every time the entrance position of the infrared light 102 is changed. Therefore, the light intensity at each exit position can be determined every time the entrance position of the infrared light 102 is changed. Consequently, brain-function measurement can be performed more accurately.

The detection unit 104 includes a plurality of detection elements constituted of high-sensitivity photomultiplier tubes or avalanche photodiodes. Thus, high-sensitivity brain-function measurement becomes possible.

Alternatively, the detection unit 104 may be a complementary metal-oxide semiconductor (CMOS) or a charge-coupled device (CCD). Thus, an image of the human head (i.e., information about, for example, the position and the orientation thereof) can be acquired together with the intensity distribution of the infrared light 102 exiting from the surface of the human head. By displaying the intensity distribution of the infrared light 102 and a visible image of the human head (which may be a near-infrared (monochrome) image) in a superimposed manner, the position of infrared-light intensity distribution in the human head (i.e., hemoglobin oxygen saturation distribution and brain function state) can be conveyed to the subject (the user) in more detail.

As a further alternative, a high-sensitivity detection unit constituted of, for example, a high-sensitivity photomultiplier tube or avalanche photodiode and a camera equipped with an inexpensive CMOS or CCD may both be provided. By using an image acquisition unit such as a camera, it becomes possible to determine whether the entrance position of the infrared light 102 overlaps an eyebrow, hair, and so on by using an image recognition technique. In addition, by also using a high-sensitivity detection unit, a high-sensitivity optical brain-function measurement apparatus is achieved.

Furthermore, if the optical brain-function measurement apparatus according to the present disclosure includes a storage unit (not shown) and makes the storage unit store past brain-function measurement results, a camera image and a brain-function measurement-result image may be stored as a set. Thus, it becomes possible to check which measurement result is whose brain-function measurement result at a later time, thereby preventing false recognition.

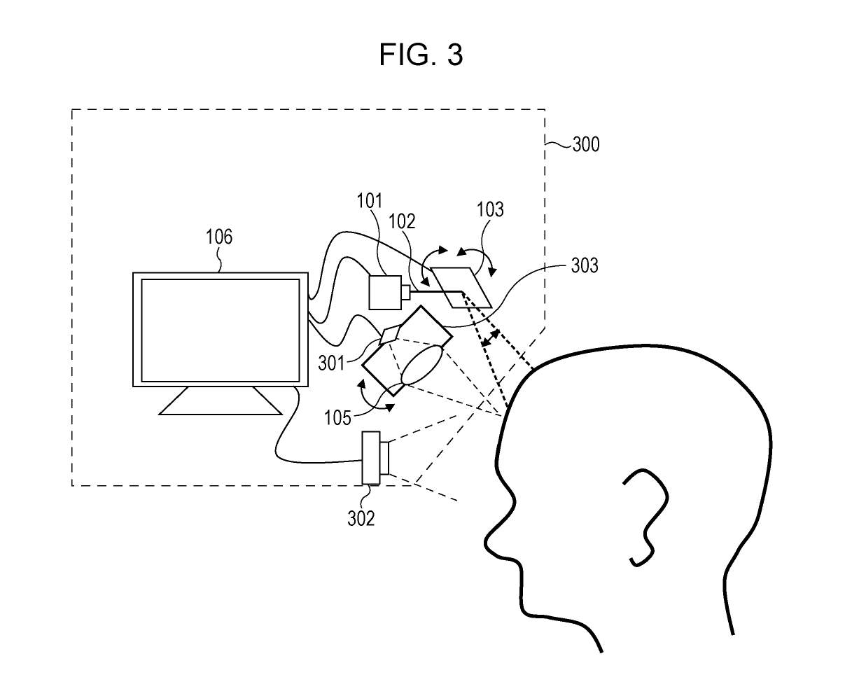

FIG. 3 illustrates another example of an optical brain-function measurement apparatus 300 according to the first embodiment.

As shown in FIG. 3, a detection unit 301 constituted of a photomultiplier tube, an avalanche photodiode, or a PIN photodiode may be used, and the light exit position, which is the field of view, may be changed by changing the angle of the detection unit 301. In this case, the optical system 105 and the detection unit 301 are combined (referred to as “detection module 303” hereinafter), and the detection module 303 is equipped with an orientation changing unit (not shown), so that the light exit position, which is the field-of-view direction, of the detection unit 301 can be shifted. A Pan-tilt adjusting unit constituted of, for example, a stepping motor is used as the orientation changing unit. Alternatively, the light exit position may be shifted by securing one of the detection unit 301 and the optical system 105 and moving the other one of the detection unit 301 and the optical system 105. Wide-angle-range brain-function measurement becomes possible by using the pan-tilt adjusting unit, whereas high-speed brain-function measurement becomes possible by moving one of the detection unit 301 and the optical system 105.

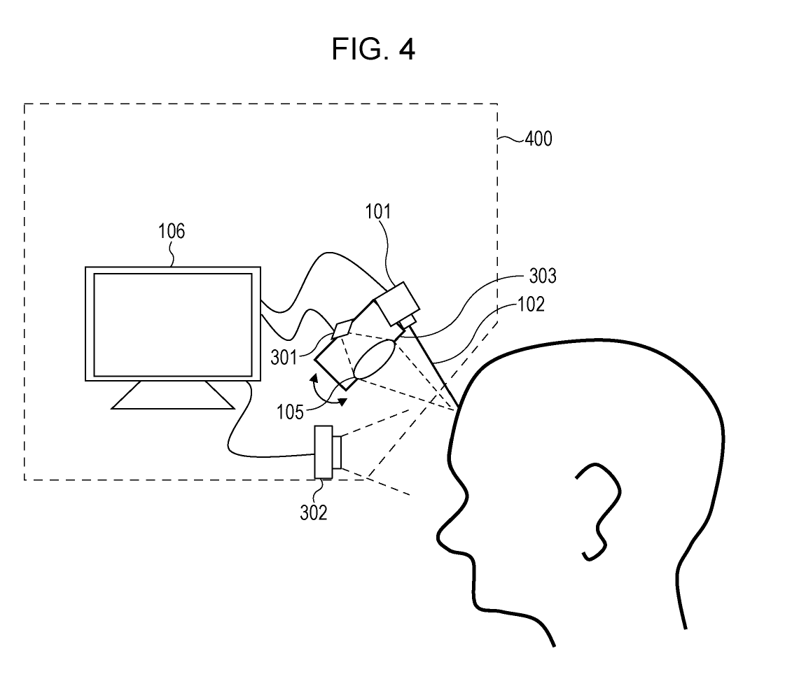

FIG. 4 illustrates another example of an optical brain-function measurement apparatus 400 according to the first embodiment.

As shown in FIG. 4, an orientation changing configuration may be achieved by combining the detection unit 301, the optical system 105, and the light source unit 101. The detection unit 301, the optical system 105, and the light source unit 101 are arranged such that infrared light 102 exiting from a position located away by a predetermined distance from a peripheral position on the surface of the head irradiated with the infrared light 102 emitted from the light source unit 101 is guided toward the detection unit 301.

In contrast to the configuration shown in FIG. 1 being the most compact and high-speed brain-function measurement apparatus, the configurations shown in FIGS. 3 and 4 have lower speed characteristics and are larger in size but allow for inexpensive brain-function measurement apparatuses.

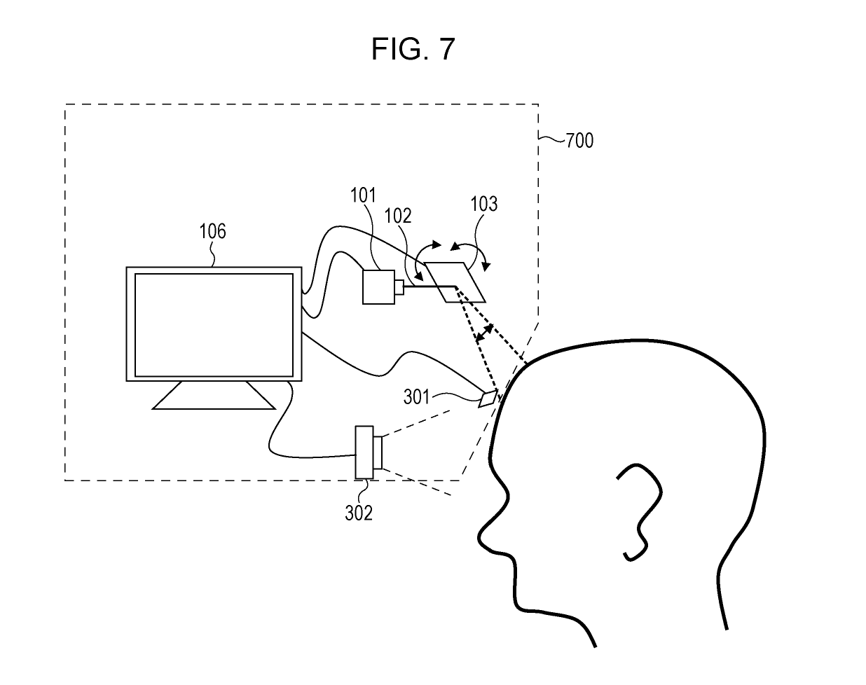

In the case where the detection unit 301 scans the light exit position with a single element, as shown in FIGS. 3 and 4 (as well as FIGS. 7, 8, and 9 to be described later), it is desirable that a head-position measuring unit 302 that measures the position of the head be provided. Thus, the position of the head can be ascertained, and the optical-path changing unit 103 and the detection unit 301 can be controlled such that the light entrance position and the light exit position are located on the head.

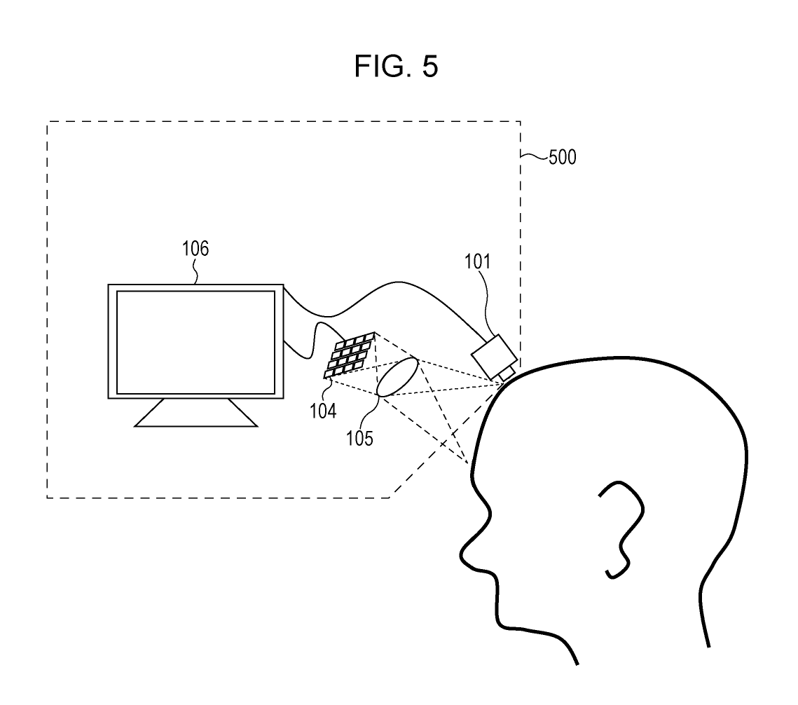

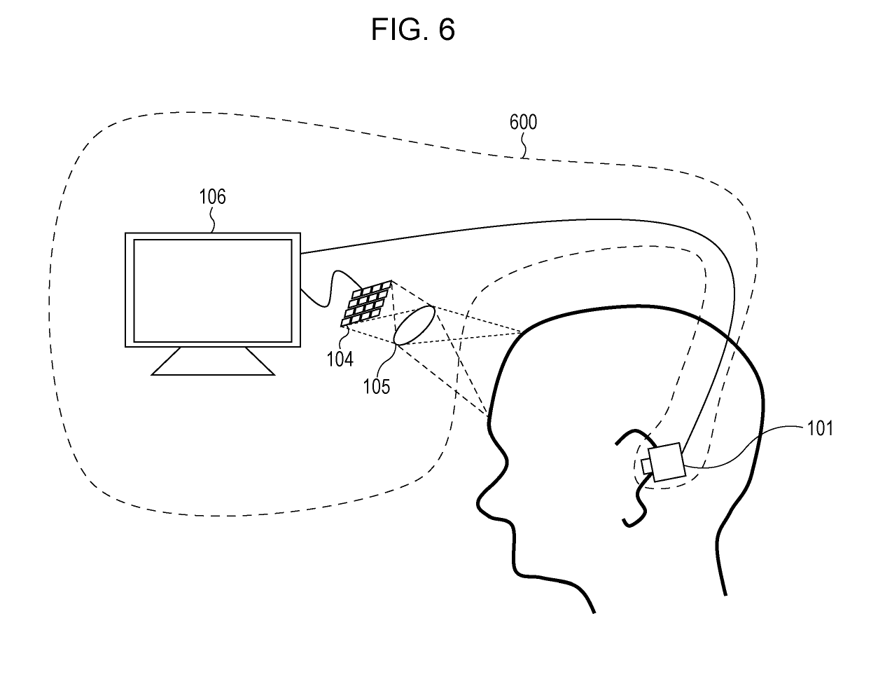

The head-position measuring unit 302 may also be provided in the case where the detection unit 104 includes a plurality of detection elements, as shown in FIG. 1 (as well as FIGS. 5 and 6 to be described later). By mapping user's-head-position information and brain-function-state information in a superimposed manner, the relationship between the positions on the head and the brain function states can be conveyed to the user in a more easily understandable manner.

The head-position measuring unit 302 may be, for example, an image acquisition unit that acquires a visible image or an infrared image. Thus, the position of the face can be ascertained based on a face recognition technique using a characteristic pattern, such as the eyes, nose, and mouth, as a mark. Furthermore, the head-position measuring unit 302 may be, for example, a distance measuring unit that measures a distance by using a time-of-flight method or a shape measuring unit that measures a shape by using a stereo camera.

Furthermore, in the configuration in which the light entrance position and the light exit position on the human head are independently determined, as in FIGS. 1 and 3, the distance between the light entrance position and the light exit position may be changed in a state where one of the light entrance position and the light exit position is fixed, so that light absorption characteristics within the brain and light absorption characteristics according to the blood flow at the surface of the head can be separated from each other. Consequently, more accurate brain-function measurement becomes possible.

Furthermore, in a state where one of the light entrance position and the light exit position is fixed, the other one of the light entrance position and the light exit position is changed without changing the distance between the light entrance position and the light exit position. Specifically, while fixing the light entrance position, the light exit position is changed such that it forms a circle around the light entrance position acting as the center, or while fixing the light exit position, the light entrance position is changed such that it forms a circle around the light exit position acting as the center. Thus, higher-sensitivity brain-function measurement becomes possible.

Furthermore, it is desirable that the infrared light 102 be transmitted and received in the aforementioned three different kinds of conditions with regard to the distance between the light entrance position and the light exit position (i.e., the aforementioned three different states with regard to the distance between the light entrance position and the light exit position). Thus, the thicknesses of the scalp and the cranium of the measurement subject can be ascertained, and the measurement result can be corrected, thereby allowing for more accurate measurement of the brain function state.

In the configuration shown in FIG. 1, for example, the light entrance position is fixed, and the intensities of infrared light from a plurality of different light exit positions can be simultaneously measured, so that high-sensitivity, higher-speed brain-function measurement can be achieved with a reduced effect of the blood flow at the surface of the head.

Furthermore, for example, it is desirable that the infrared light 102 be transmitted and received by fixing the light entrance position and changing the light exit position such that the distance between the light entrance position and the light exit position changes at intervals of 2 mm to 0.5 mm. Thus, based on the infrared-light intensity detected by the detection unit 104, if the light quantity of infrared light significantly changes at a short distance of about 2 mm to 0.5 mm, it can be determined that the area corresponding to the light exit position is an area in which an artery or a vein exists under the scalp, so that the light quantity of infrared light exiting from this light exit position may be not used for the brain-function measurement.

Alternatively, the infrared light 102 may be transmitted and received by fixing the light exit position and changing the light entrance position such that the distance between the light entrance position and the light exit position changes at intervals of 2 mm to 0.5 mm. Thus, based on the infrared-light intensity detected by the detection unit 104, if the light quantity of infrared light significantly changes at a short distance of about 2 mm to 0.5 mm, it can be determined that the area corresponding to the light entrance position is an area in which an artery or a vein exists under the scalp. Therefore, the optical-path changing unit 103 can be controlled such that this light entrance position is avoided and the infrared light 102 is made to enter from a position which is set away from the artery or the vein in the scalp.

In other words, since the position of an artery or a vein in the scalp can be ascertained, at least one of the light entrance position and the light exit position can be set away from the artery or the vein in the scalp, thereby achieving increased accuracy for the brain-function measurement.

As an alternative to the above-described method, brain-function measurement that is not affected by the position of an artery or a vein can be achieved, for example, by averaging transmission and reception results of the infrared light 102 at a plurality of light entrance positions or a plurality of light exit positions or by removing maximum data and minimum data.

Although the method of ascertaining the position of an artery or a vein allows for higher accuracy, the latter methods, namely, the averaging method and the maximum-minimum removal method, are more advantageous in terms of measurement speed.

### Second Embodiment

A second embodiment relates to an optical brain-function measurement apparatus that is used by bringing the light source unit into contact with the human body.

By bringing the light source unit into contact with the human body, higher-output infrared light can be radiated so that a higher-sensitivity optical brain-function measurement apparatus can be achieved. However, since the position of the light source unit cannot be changed, the function state at an arbitrary position within the brain can be measured by changing only the exit position.

FIG. 5 illustrates an optical brain-function measurement apparatus 500 according to the second embodiment.

As shown in FIG. 5, in the optical brain-function measurement apparatus 500, the light source unit 101 is brought into contact with the scalp. With this scalp contact type, the output of light entering the human head from the light source unit 101 can be increased, so that higher-sensitivity brain-function measurement becomes possible.

The detection unit 104 may have a configuration identical to that in FIGS. 3 and 4.

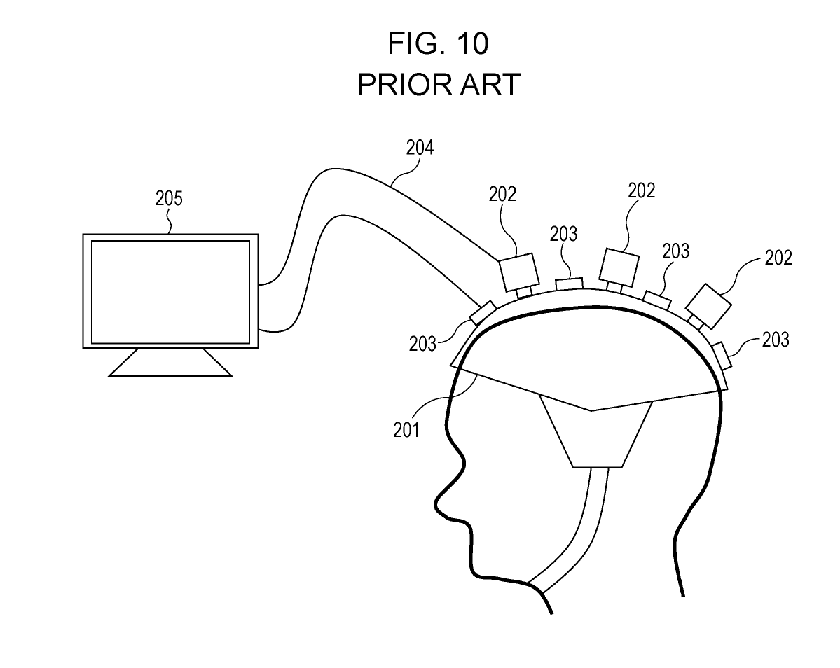

In order to secure the light source unit 101 into contact with the scalp, the wearable member 201 shown in FIG. 10 may be used as a securing unit.

FIG. 6 illustrates another example of an optical brain-function measurement apparatus 600 according to the second embodiment.

As shown in FIG. 6, the optical brain-function measurement apparatus 600 is used by fitting the light source unit 101 into user's ear. Infrared light is output within the earhole, and the infrared light exiting through the scalp is measured, so that a signal according to light absorbance within the brain from the ear to the scalp can be measured. In other words, this is desirable since the function state in a deep area within the brain can also be measured.

Likewise, the light source unit 101 may be fitted into user's nostril or mouth. However, the method of fitting the light source unit 101 into user's earhole is the most desirable since this method allows for a stable fit position and does not hinder breathing.

In the case of the optical brain-function measurement apparatus 600, the detection unit 104 may have a configuration identical to that in FIGS. 3 and 4.

When the configurations shown in FIGS. 5 and 6 are compared, the configuration shown in FIG. 5 is desirable in terms of higher sensitivity, whereas the configuration shown in FIG. 6 is desirable in terms of the ability to measure a deep area within the brain.

### Third Embodiment

A third embodiment relates to an optical brain-function measurement apparatus according to the present disclosure that is used by bringing the detection unit into contact with the human body.

By bringing the detection unit into contact with the human body, the effect of ambient light can be further reduced so that a higher-sensitivity optical brain-function measurement apparatus can be achieved. However, since the position of the detection unit cannot be changed, the function state at an arbitrary position within the brain can be measured by changing only the entrance position.

FIG. 7 illustrates an optical brain-function measurement apparatus 700 according to the third embodiment.

As shown in FIG. 7, in the optical brain-function measurement apparatus 700, the detection unit 301 is attached so as to be secured onto the head. The number of detection units to be attached may be two or more.

In order to attach and secure the detection unit 301 onto the head, the wearable member 201 shown in FIG. 10 may be used as a securing unit.

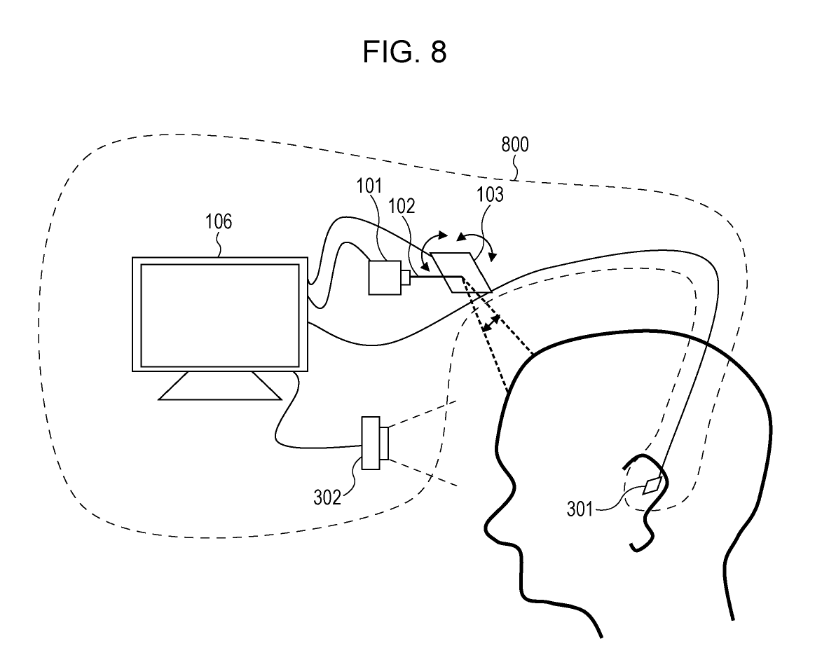

FIG. 8 illustrates another example of an optical brain-function measurement apparatus 800 according to the third embodiment.

As shown in FIG. 8, the optical brain-function measurement apparatus 800 is used by fitting the detection unit 301 into user's ear. Infrared light enters through the scalp, and the infrared light is measured within the earhole, so that a signal according to light absorbance within the brain from the ear to the scalp can be measured. In other words, this is desirable since the function state in a deep area within the brain can also be measured.

Likewise, the detection unit 301 may be fitted into user's nostril or mouth. However, the method of fitting the detection unit 301 into user's earhole is the most desirable since this method allows for a stable fit position and does not hinder breathing.

When the configurations shown in FIGS. 7 and 8 are compared, the configuration shown in FIG. 7 is desirable in terms of higher sensitivity, whereas the configuration shown in FIG. 8 is desirable in terms of the ability to measure a deep area within the brain.

Needless to say, similar advantages are exhibited in the second and third embodiments by using a configuration similar to that in the first embodiment.

The optical brain-function measurement apparatus according to each of the above embodiments may command the subject (the user) to raise hair covering the forehead by means of a human interface, such as a voice or an image. Thus, higher-sensitivity frontal-lobe brain-function measurement becomes possible.

For example, this will be described with reference FIG. 3 as an example.

If the position of the head measured by the head-position measuring unit 302 is deviated from a predetermined position, the user may be informed of the situation by means of a voice, such as “please bring your head closer” or “please move your head to the right”, or by displaying a message on an image display unit. This allows for measurement of user's brain function more accurately.

If the head-position measuring unit 302 is the image acquisition unit, the head-position measuring unit 302 may measure how the hair is covering the forehead. If the hair is covering the forehead, the user may be informed of the situation by means of a voice, such as “please raise your hair” or “the hair on your forehead is impeding measurement”, or by displaying a message on the image display unit. This allows for measurement of user's brain function more accurately.

Furthermore, if the measurement accuracy is low, the user may be informed of the reason therefor similarly by means of a voice or image display unit, such as “there is too much dust in the measurement environment” or “the intensity of ambient light (sunlight) is too high”. Thus, the user can ascertain that the measurement accuracy is low, so that a certain measure can be taken.

Furthermore, as shown in FIGS. 5 to 8, when one of the light source unit 101 and the detection unit 104 or 301 is of a contact type, the light source unit 101 or the detection unit 104 or 301 may be secured to the head by using, for example, eyeglasses or a band as securing unit.

Furthermore, it is desirable that a skin-temperature measuring unit that measures the temperature (i.e., skin temperature) of the surface of the head near the entrance and exit positions of the infrared light 102 be provided. Moreover, it is desirable that a skin-condition measuring unit that measures the condition (i.e., skin condition) of the surface of the head near the entrance and exit positions of the infrared light 102 be provided. The skin-condition measuring unit may be, for example, a skin-moisture measuring unit that measures skin moisture on the surface of the head near the entrance and exit positions of the infrared light 102. For example, the skin-temperature measuring unit for measuring the temperature near the entrance position where the infrared light 102 enters the surface of the head is desirably provided at positions adjacent to the light source unit 101.

For example, the skin-moisture measuring unit for measuring the condition near the entrance position where the infrared light 102 enters the surface of the head is desirably provided at positions adjacent to the light source unit 101.

Furthermore, for example, the skin-temperature measuring unit for measuring the temperature of the surface of the head near the exit position where the infrared light 102 exits the surface of the head is desirably provided at positions adjacent to the detection unit.

For example, the skin-moisture measuring unit for measuring the condition of the surface of the head near the exit position where the infrared light 102 exits the surface of the head is desirably provided at positions adjacent to the detection unit.

Consequently, with regard to surface scattering of the infrared light 102 that fluctuates depending on the skin temperature and the skin condition, the effect of scatter reflections at the epidermal layer and the dermal layer can be corrected. Thus, scatter and transmission characteristics within the brain can be ascertained (i.e., brain-function measurement can be performed) more accurately.

For example, when the skin temperature and the skin moisture percentage change, light absorption characteristics change in accordance with moisture in the epidermal layer and the dermal layer. With the above-described configuration, this effect can be reduced.

As the skin-temperature measuring unit, a non-contact irradiation temperature measuring unit, such as a thermopile or a bolometer, is used. Although the skin-temperature measuring unit may be of a contact type, such as a thermistor or a thermocouple, since an area with which a thermistor or a thermocouple used as the skin-temperature measuring unit is brought into contact cannot be irradiated with light and thus becomes an area where the brain function cannot be measured, the non-contact irradiation temperature measuring unit is desirably used as the skin-temperature measuring unit.

The skin-moisture measuring unit may be of a contact type that calculates skin moisture based on electric conductivity, but is desirably of a non-contact type due to similar reasons described above. For example, skin-moisture measurement may be performed by utilizing light absorption characteristics in a near-infrared to far-infrared region. For example, by emitting a light beam with a wavelength near 1.5 μm and a light beam with a wavelength near 1.4 μm from the light source unit 101, skin-moisture measurement of the epidermal layer can be performed simultaneously with the brain-function measurement. Needless to say, the wavelengths of the light beams may be 1.5 μm and 1.6 μm, or 1.55 μm and 1.64 μm. By using light beams of a plurality of wavelengths having different light absorbance with respect to water, the moisture percentage can be estimated.

Alternatively, wavelengths with high water absorbance, such as a mid-infrared region or a far-infrared region near 6 μm or near 3 μm, may be used.

### Fourth Embodiment

A fourth embodiment relates to a portable, compact optical brain-function measurement apparatus in which the light source unit and the detection unit are combined.

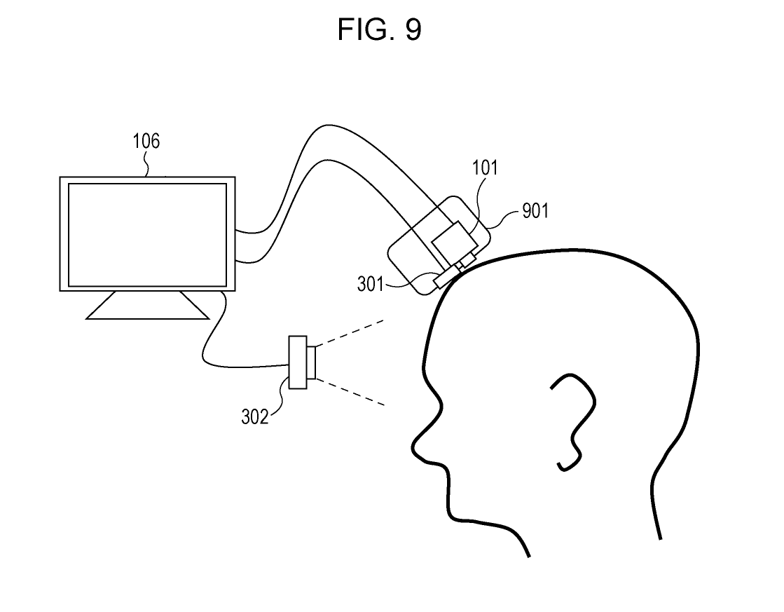

As shown in FIG. 9, the optical brain-function measurement apparatus includes at least one of the light source unit 101 and the detection unit 301. The light source unit 101 and the detection unit 301 are attached to an optical transmission-reception probe 901 such that the distance between the two units does not change.

In the optical brain-function measurement apparatus according to the fourth embodiment, both the light source unit 101 and the detection unit 301 are of a contact type. The user can hold the optical transmission-reception probe 901 and move it to an arbitrary position, so that brain-function measurement at the arbitrary position becomes possible. Moreover, measurement can be performed while changing the light entrance and exit positions.

However, the accuracy for ascertaining the relationship between the light entrance and exit positions and the brain function is lower than that in the configurations according to the first to third embodiments, and the configurations according to the first to third embodiments are more advantageous in terms of the speed for measuring the entire measurement subject.

Furthermore, although not shown, by providing a plurality of detection units with different distances from the light source unit 101, higher-sensitivity brain-function measurement becomes possible.

Alternatively, by providing a plurality of detection units with an equal distance from the light source unit 101, the light absorbance of the blood flow at the scalp surface and the light absorbance of the blood flow within the brain can be separately determined, whereby higher-sensitivity brain-function measurement becomes possible.

Furthermore, in the fourth embodiment, the skin-temperature measuring unit and the skin-moisture measuring unit may be provided at positions adjacent to the light source unit 101 and the detection unit 301. Thus, the advantages described above can be achieved.

Furthermore, the image display unit 1007 accompanying the main device 106 may display information such as the measured skin temperature and the measured skin moisture. Since the human body can ascertain the skin temperature and the skin moisture simultaneously with the brain-function measurement, an optical brain-function measurement apparatus that is practical for health and cosmetic management purposes can be provided.

The optical brain-function measurement apparatus according to any one of the first to fourth embodiments may be equipped in an apparatus having other various kinds of functions.

For example, a vehicle having the optical brain-function measurement apparatus according to any one of the first to fourth embodiments provided in the driver seat is permissible. The degree of sleepiness of the driver may be estimated, and ventilation within the vehicle (carbon-dioxide (CO₂) concentration control) may be performed based on the estimation result.

Furthermore, a desk (equipped with a light) or a desk light equipped with the optical brain-function measurement apparatus according to any one of the first to fourth embodiments is also permissible. The degree of concentration or the degree of sleepiness of the measurement subject may be estimated, and the degree of concentration may be enhanced by controlling the intensity of the light in accordance with the estimation result.

An air-quality (i.e., CO₂ concentration, humidity, temperature, and concentration of other components) adjusting unit equipped with the optical brain-function measurement apparatus according to any one of the first to fourth embodiments is also permissible. The air-quality adjusting unit adjusts the air quality by estimating the degree of concentration, the degree of sleepiness, or the emotion.

An audio-visual (AV) apparatus equipped with the optical brain-function measurement apparatus according to any one of the first to fourth embodiments is also permissible. By selecting music, an image, and so on in accordance with the degree of sleepiness or the emotion, an environment more suitable for each individual can be provided.

A factory line apparatus equipped with the optical brain-function measurement apparatus according to any one of the first to fourth embodiments is also permissible. Optimal working-hour adjustment can be performed, such as stopping a line and providing a rest period in accordance with the degree of concentration of the workers, or variations in load on the workers may be ascertained so that, for example, alternation of the working process can be suggested by means of a voice or by displaying an image.

By installing the optical brain-function measurement apparatus according to any one of the first to fourth embodiments inside a meeting room, the degree of sleepiness or emotional excitement of participants may be estimated so as to control air-conditioning and lighting in the meeting room or to prompt the participants to end the meeting by means of, for example, a voice. Thus, a wasteful meeting by a participant or participants lacking concentration can be prevented.

Furthermore, a television set equipped with the optical brain-function measurement apparatus according to any one of the first to fourth embodiments is also permissible. An advertisement effect can be enhanced by, for example, selecting an appropriate commercial in accordance with the emotional state.

In each of the configurations described above, the optical brain-function measurement apparatus and the apparatus having another function (such as a vehicle or a desk) may be separate apparatuses if both apparatuses are equipped with a communication unit.

Furthermore, in the above description, in order to estimate the state of the measurement subject (such as the degree of concentration, the degree of sleepiness, the comfort level, or the discomfort level), the optical brain-function measurement apparatus according to the present disclosure may be used in combination with, for example, an electroencephalograph, a heart rate meter, a blood pressure meter, and a laser speckle blood flow meter so that the state can be estimated more accurately.

Furthermore, although the present disclosure relates to an optical brain-function measurement apparatus, the concentration distribution of oxyhemoglobin and deoxyhemoglobin in a part of the human body other than the brain can also be measured with a similar configuration.

Although the optical brain-function measurement apparatus according to the present disclosure has been described above, it is also possible to measure a cosmetic condition with a similar configuration. Specifically, for example, the optical path of infrared light emitted from the light source unit 101 is changed by the optical-path changing means 103 so that the infrared light is radiated onto an area under one's eye.

The infrared light radiated onto the under-eye area is diffusely-reflected within the human head and subsequently exits the head.

Of the infrared light radiated onto the under-eye area, light entering mainly through the irradiation position (i.e., the under-eye area) and then exiting the face again after passing between the under-eye area (i.e., skin) and the bone located therebelow is detected by the detection unit 104.

A portion of this infrared light passing between the under-eye area (i.e., skin) and the bone located therebelow is absorbed by passing through a blood vessel. For example, the infrared light is absorbed by deoxyhemoglobin contained in the blood flowing through the blood vessel. The amount of deoxyhemoglobin contained in the blood varies depending on, for example, the health condition of a subject.

Therefore, by using the detection unit 104 to measure the quantity of infrared light exiting the face for a specific time period, the amount of deoxyhemoglobin flowing through the blood vessel per unit time or the concentration of deoxyhemoglobin contained in the blood can be estimated. Thus, the health condition (cosmetic condition) of the subject can be measured based on the estimated amount or concentration of deoxyhemoglobin.

In this case, the distance between the infrared-light irradiation position in the under-eye area and the position from which the infrared light to be detected by the detection unit 104 exits the face is desirably short (for example, between 0.3 cm and 2.0 cm). This is because, if the distance is short, it can be considered that the infrared light exiting the face is the infrared light having passed between the under-eye area (skin) and the bone located therebelow.

Therefore, it is desirable that the infrared-light irradiation position in the under-eye area be controlled by the optical-path changing means 103.

Alternatively, the position from which the infrared light to be detected by the detection unit 104 exits the face is desirably controlled by using the optical system 105.

By controlling at least one of the infrared-light irradiation position in the under-eye area and the position from which the infrared light to be detected by the detection unit 104 exits the face, the distance between these positions can be controlled within the range between 0.3 cm and 2.0 cm.

Furthermore, although the present disclosure relates to an optical brain-function measurement apparatus, the measurement subject may be a plant or a food material, such as a fruit, a vegetable, or meat. In this case, componential analysis within the measurement subject can be performed while an effect caused by the surface state thereof can be reduced.

It is needless to say that advantages can be exhibited by the respective configurations described in this specification.

### MODIFICATIONS

Configurations and modifications of an optical brain-function measurement apparatus according to the present disclosure will be described below.

An optical brain-function measurement apparatus according to an embodiment of the present disclosure includes a light source unit that generates infrared light to be radiated onto a human head; a detection unit that detects the infrared light which is diffusely-reflected within the human head and which is exited from one or more head position; an optical system that guides the infrared light emitted from the light source unit toward the human head and that controls an infrared-light irradiation position on a surface of the human head. The light source unit is a light source of a non-contact type that does not contact to the human or the detection unit is a detector of a non-contact type that does not contact to the human. The one or more head positions detected by the detection unit include at least one position that is different from the infrared-light irradiation position controlled by the optical-path changing unit.

According to this configuration, an optical brain-function measurement apparatus that achieves brain-function measurement at an arbitrary position of the human head can be provided.

In the optical brain-function measurement apparatus according to the embodiment of the present disclosure, the detection unit is the detector of the non-contact type and includes a plurality of detection elements that detect the infrared light exiting from the one or more head positions.

In the optical brain-function measurement apparatus according to the embodiment of the present disclosure, the detection unit is the detector of the non-contact type and includes a detecting position changing unit that changes the one or more head positions from which the infrared light to be detected by the detection unit exits.

In the optical brain-function measurement apparatus according to the embodiment of the present disclosure, the light source unit is the light source of the non-contact type, and the optical system includes the optical-path changing unit that changes the infrared-light irradiation position on the surface of the human head.

The optical brain-function measurement apparatus according to the embodiment of the present disclosure further includes skin-temperature measuring unit that measures a skin temperature near the infrared-light irradiation position on the surface of the human head.

The optical brain-function measurement apparatus according to the embodiment of the present disclosure further includes skin-moisture measuring unit that measures skin moisture near the infrared-light irradiation position.

The optical brain-function measurement apparatus according to the embodiment of the present disclosure further includes a head-position measuring unit that measures the one or more head positions from which the infrared light to be detected by the detection unit exits.

In the optical brain-function measurement apparatus according to the embodiment of the present disclosure, the infrared-light irradiation position is controlled based on a detection result of the detection unit.

Although the optical brain-function measurement apparatus according to the present disclosure has been described above, the configurations described in this specification are examples, and various modifications are possible so long as they do not depart from the scope of the present disclosure.

The present disclosure is suitably applicable to an optical brain-function measurement apparatus that measures brain activity by radiating light onto a human head and measuring light that has passed through the human brain.

What is claimed is:

1. An optical brain-function measurement apparatus comprising:
a light source unit that generates infrared light to be radiated onto a human head;
a detection unit that detects the infrared light which is diffusely-reflected within the human head and which is exited from one or more head position; and
an optical system that guides the infrared light emitted from the light source unit toward the human head and that controls an infrared-light irradiation position on a surface of the human head,
wherein the light source unit is a light source of a non-contact type that does not contact to the human or the detection unit is a detector of a non-contact type that does not contact to the human, and
wherein the one or more head positions detected by the detection unit includes at least one position that is different from the infrared-light irradiation position controlled by the optical system.

2. The optical brain-function measurement apparatus according to claim 1,
wherein the detection unit is the detector of the non-contact type and includes a plurality of detection elements that detect the infrared light exiting from the one or more head positions.

3. The optical brain-function measurement apparatus according to claim 1,
wherein the detection unit is the detector of the non-contact type and includes a detecting position changing unit that changes the one or more head positions from which the infrared light to be detected by the detection unit exits.

4. The optical brain-function measurement apparatus according to claim 1,
wherein the light source unit is the light source of the non-contact type, and
wherein the optical system includes optical-path changing unit that changes the infrared-light irradiation position on the surface of the human head.

5. The optical brain-function measurement apparatus according to claim 1, further comprising:
skin-temperature measuring unit that measures a skin temperature near the infrared-light irradiation position.

6. The optical brain-function measurement apparatus according to claim 1, further comprising:
skin-moisture measuring unit that measures skin moisture near the infrared-light irradiation position.

7. The optical brain-function measurement apparatus according to claim 1, further comprising:
a head-position measuring unit that measures the one or more head positions from which the infrared light to be detected by the detection unit exits.

8. The optical brain-function measurement apparatus according to claim 4,
wherein the infrared-light irradiation position is controlled based on a detection result of the detection unit.
